(19) United States
(12) Patent Application Publication
(10) Pub. No.: US 2021/0154100 A1
(43) Pub. Date: May 27, 2021

(54) Pet Treat Pocket

(71) Applicant: Shauna Angelidis, Houston, TX (US)

(72) Inventor: Shauna Angelidis, Houston, TX (US)

(21) Appl. No.: 17/011,045

(22) Filed: Sep. 3, 2020

Related U.S. Application Data
(60) Provisional application No. 62/940,342, filed on Nov. 26, 2019.

(51) Int. Cl. A61J 7/00; A01K 13/00; A61J 1/03; A23K 40/30; A23K 20/28; A23K 20/147; A61K 9/00

(52) U.S. Cl. CPC A61J 7/0015; A01K 13/003; A61J 1/03; A61K 9/0056; A23K 20/28; A23K 20/147; A23K 40/30

(57) ABSTRACT

A pet treat pocket is provided. The pet treat pocket has a flexible member including a pocket disposed on a top surface of the flexible member. The pocket defines an interior volume and the interior volume is sized to encapsulate a pill. An exterior length of the pocket is less than a length of the flexible member, and an exterior width of the pocket is less than a width of the flexible member such that the flexible member extends beyond the exterior dimensions of the pocket forming a lip surrounding a circumference of the pocket. The flexible member and the pocket are both composed of an ingestible and pet-safe material. The ingestible and pet-safe material can adhere to itself such that the flexible material can be molded to completely envelope the pocket and maintain a sealed configuration.

### CROSS REFERENCE TO RELATED APPLICATIONS

This application claims the benefit of U.S. Provisional Application No. 62/940,342 filed on Nov. 26, 2019. The above identified patent application is herein incorporated by reference in its entirety to provide continuity of disclosure.

### BACKGROUND OF THE INVENTION

The present invention relates to pet treats and medications. More particularly, the present invention provides for a pet-safe ingestible material which includes a pocket. The present invention can be offered by itself as a healthy treat to a pet, or a pill can be inserted into the pocket and the material hand molded to encapsulate the pill within the pocket. The pet-safe ingestible material can block or mask odors from the pill such that a pet in unable to detect the presence of the pill within the pocket.

Many people have pets who require medication for maintenance or improvement of their health or well-being. However, such medications are often challenging to feed to these pets. Some pets refuse direct ingestion of medications. In such circumstances, it may be difficult or impossible to administer the medications. Some pets are able to detect medications by smell even when hidden within food and may eat around the medication.

Devices have been disclosed in the known art that relate to pet treats and medications. These include devices that have been patented and disclosed in patent application publications. However, the devices in the known art have several drawbacks. Some devices include an opening in which a pill can be placed. However, these devices may not secure the pill and the pill can fall out. Other devices may not prevent the pet from detecting the presence of the pill, either by sight or smell. Other devices may be ingestible but contain chemicals and ingredients that are harsh on the pet's digestive system and do not provide much nutritional value.

The present invention substantially diverges in design elements from the known art and consequently it is clear that there is a need in the art for an improvement to existing pet treat and medication devices. In this regard the present invention substantially fulfills these needs.

### SUMMARY OF THE INVENTION

In view of the foregoing disadvantages inherent in the known types of pet treat and medication devices now present in the art, the present invention provides a pet-safe ingestible material which includes a pocket. The present invention can be offered by itself as a healthy treat to a pet, or a pill can be inserted into the pocket and the material hand molded to encapsulate the pill within the pocket. The pet-safe ingestible material can block or mask odors from the pill such that a pet in unable to detect the presence of the pill within the pocket. The present pet treat pocket comprises a flexible member including a pocket disposed on a top surface of the flexible member. The pocket defines an interior volume and the interior volume is sized to encapsulate a pill. An exterior length of the pocket is less than a length of the flexible member, and an exterior width of the pocket is less than a width of the flexible member such that the flexible member extends beyond the exterior dimensions of the pocket forming a lip surrounding a circumference of the pocket. The flexible member and the pocket are both composed of an ingestible and pet-safe material. The ingestible and pet-safe material can adhere to itself such that the flexible material can be molded to completely envelope the pocket and maintain a sealed configuration.

Other objects, features, and advantages of the present invention will become apparent from the following detailed description taken in conjunction with the accompanying drawings.

### BRIEF DESCRIPTION OF THE DRAWINGS

Although the characteristic features of this invention will be particularly pointed out in the claims, the invention itself and manner in which it may be made and used may be better understood after a review of the following description, taken in connection with the accompanying drawings wherein like numeral annotations are provided throughout.

FIG. 1 shows a front perspective view of an embodiment of the pet treat pocket being used to receive a pill.

FIG. 2 shows a front perspective view of a row of seven pet treat pockets in an embodiment of the pet treat pocket.

FIG. 3 shows a front perspective view of an embodiment of the pet treat pocket illustrating how the pet treat pocket can be folded to enrobe a pill.

FIG. 4 shows a chart of a method of use of an embodiment of the pet treat pocket.

### DETAILED DESCRIPTION OF THE INVENTION

Reference is made herein to the attached drawings. Like reference numerals are used throughout the drawings to depict like or similar elements of the pet treat pocket. For the purposes of presenting a brief and clear description of the present invention, a preferred embodiment will be discussed as used for the pet treat pocket. The figures are intended for representative purposes only and should not be considered to be limiting in any respect.

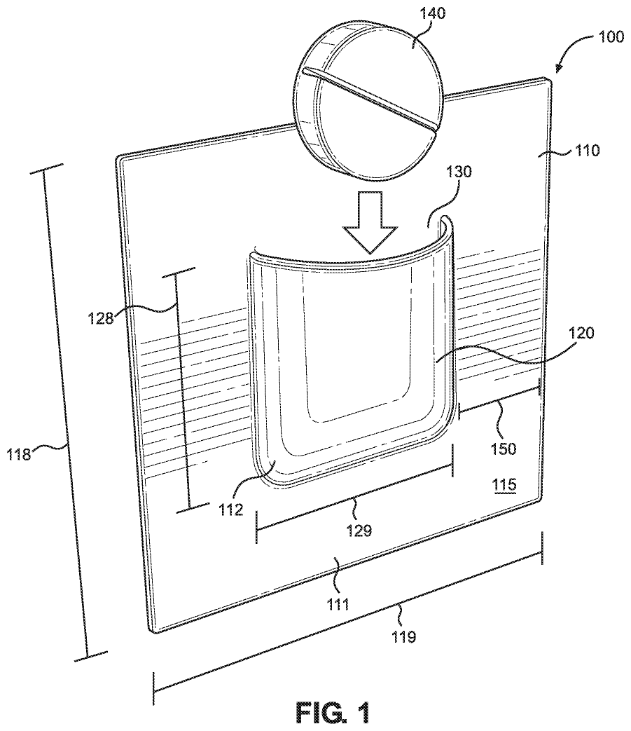

Referring now to FIG. 1, there is shown a front perspective view of an embodiment of the pet treat pocket being used to receive a pill. The pet treat pocket 100 comprises a flexible member 110 including a pocket 120 disposed on a top surface 115 of the flexible member 110. In some embodiments, the flexible member 110 and the pocket 120 are composed of a single layer of a material. In such embodiments, production and manufacturing costs are cut to a minimum and the likelihood of the flexible member 110 detaching is eliminated. In other embodiments, the flexible member 110 is a first layer 111 and the pocket 120 is a second layer 112 disposed on a top surface 115 of the flexible member 110. Such embodiments provide easy manufacturing and assembly of the pet treat pocket 100.

The pocket 120 defines an interior volume 130. The interior volume 130 is sized to encapsulate at least one pill 140. As one object of the present invention is to hide and mask the appearance and odors from the pill 140, one of ordinary skill in the art will understand the interior volume 130 dimensions can vary from one embodiment to another based on the size of the pill 140. In the shown embodiment, the pocket 120 is connected to the flexible member 110 on three of four sides. The fourth, open side, enables a user to slide the pill 140 into the pocket 120. In the shown embodiment, the entirety of the fourth side is open, however, in other embodiments, only a portion of the fourth side is open with the rest of the fourth side connected to the flexible member 110. In the shown embodiment, the pocket 120 comprises an arcuate shape in order to facilitate easier insertion of the pill 140 into the pocket 120.

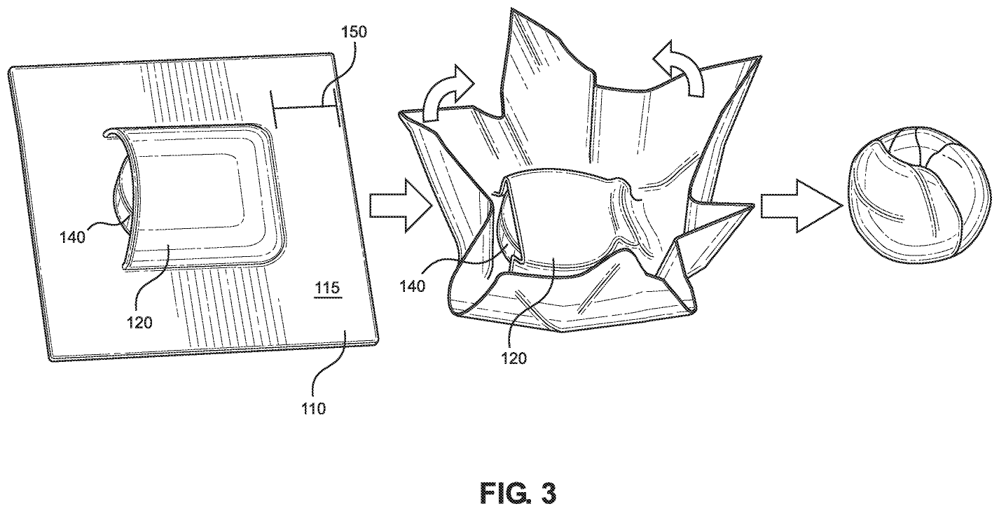

An exterior pocket length 128 is less than a flexible member length 118, and an exterior pocket width 129 is less than a flexible member width 119 such that the flexible member 110 extends beyond the exterior dimensions of the pocket 120, thereby forming a lip 150. In the shown embodiment, the lip 150 surrounds a circumference of the pocket 120. In the preferred embodiment, the lip 150 is sized to completely enrobe an entirety of the pocket 120 when the flexible member 110 is molded over the pocket 120 (as shown in FIG. 3).

The flexible member 110 and the pocket 120 are both composed of an ingestible and pet-safe material. In the preferred embodiment, the ingestible and pet-safe material is organic and is palatably flavored in order to minimize inorganic compounds ingested and to promote a pet to eat the pet treat pocket by the nature of its flavor. In some embodiments, the ingestible and pet-safe material is a thin gelatin-based material. An object of the present invention is to enrobe a pill 140 in the pet treat pocket 100 and the thin gelatin-based material enables the invention to be easily folded and molded. In the preferred embodiment, the ingestible and pet-safe material is malleable by hand thereby enabling a user to manipulate and mold the material without any additional tools.

In a preferred formulation, the ingestible and pet-safe material comprises montmorillonite clay and a binding material, such as gelatin. The montmorillonite clay provides significant health benefits to the pet in that it offers 67 different minerals including: calcium, iron, magnesium, potassium, and the like. The montmorillonite clay also has a detoxifying agent which eliminates toxins from the body. It has proven to aid skin and ear allergies, digestive disturbances, thyroid, adrenal and pancreatic disorders, seizures, kidney disease and liver issues. It also relieves arthritic pain, skin infections, allergic reactions, vomit, and diarrhea. The clay can also draw out toxins, allergens, parasites, and bacteria while detoxifying the intestinal tract of the pet. Relatedly, calcium bentonite has been used in similar clays as an anti-caking agent which is added to the formulation to absorb excess moisture and aids in preventing foods from clumping. The present invention does not utilize such anti-caking agents, and thus in the preferred embodiments, the ingestible and pet-safe material does not compromise calcium bentonite.

The ingestible and pet-safe material is configured to adhere to itself. As mentioned previously, an object of the present invention is to enrobe and encapsulate a pill 140 within the invention. As the ingestible and pet-safe material can adhere to itself, upon the material being folded and molded into a desired geometry, the geometry is maintained as a function of this self-adherence. Of particular note is the fact that by virtue of the pocket 120 being disposed on the top surface 115 of the flexible member 110, in various embodiments, the present device provides a double enrobing of the pill 140; the first from the pocket 120 itself, and the second from wrapping the lip 150 and the remaining portions of the flexible member 110 around the pill 140.

Another object of the present invention is to hide, mask, block, or otherwise prevent the pet from detecting the presence of the pill 140 with the pet treat pocket 100. Some pets have difficulty taking pills, typically by virtue of the smell and taste of the pill 140. Thus, one object of the present invention is to eliminate the odor such that the pet will ingest the pet treat pocket without problem. In one embodiment, upon the pill 140 being encapsulated by the pocket 120, the ingestible and pet-safe material blocks odors emanating from the pill 140 from escaping the pocket 120. In this manner, a pet will have a harder time detecting the presence of the pill 140. In another embodiment, upon the pill 140 being encapsulated by the pocket 120, the ingestible and pet-safe material masks odors emanating from the pill 140 from escaping the pocket 120, such as by providing a stronger scent that covers the odors emanating from the pill 140. In this manner, the pet also has a harder time detecting the presence of the pill 140 as the alternate odors can cover the odors emanating from the pill 140. Additionally, by virtue of the double enrobing of the pill 140, odors emanating from the pill 140 are similarly masked or blocked.

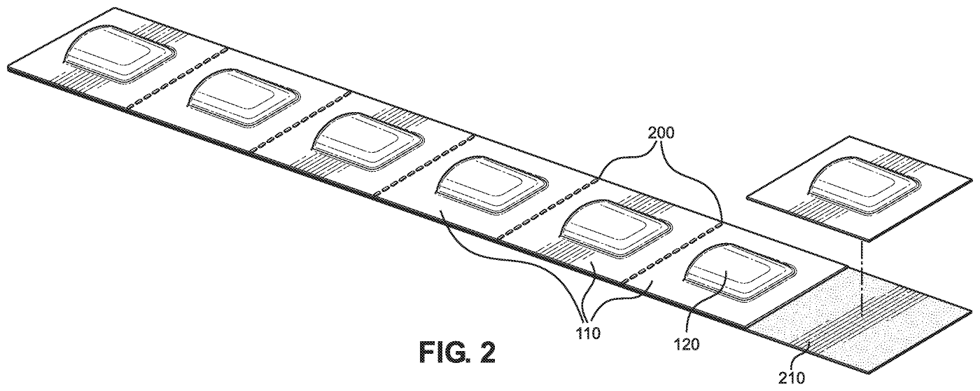

Referring now to FIG. 2, there is shown a front perspective view of a row of seven pet treat pockets in an embodiment of the pet treat pocket. In the shown embodiment, a plurality of pockets 120 are disposed on the flexible member 110. Further, in the shown embodiment, seven (7) pockets 120 are disposed on the flexible member 110, such that a user can plan to utilize a single pocket 120 for each day of the week. As some pets require multiple pills daily, the pocket 120 can be sized to accommodate multiple pills at once. In this manner, a single pocket 120 can be filled with desired medication and detached from the flexible member 110 each day of the week. In the shown embodiment, at least one perforation 200 separates at least one of the plurality of pockets 120 from a neighboring pocket 120 of the plurality of pockets 120. As pills may be of different sizes, or multiple doses of pills required per day, in such an embodiment, a user can selectively choose the number of pockets 120 required and separate those pockets 120 for use throughout the day/week/desired timeframe.

In the shown embodiment, a backing material 210 is disposed along a rear surface of the flexible member 110. The backing material 210 is removably secured to the rear surface of the flexible member 110 such that when a user desires to utilize a given pocket 120, they can remove the backing material 210 to expose the rear surface of the flexible material 110. The backing material 210 provides the benefit of reducing the amount of exposure that the flexible material 110 has to the environment, and thus protects the flexible material 110 from becoming dirty, contaminated, or the like. Additionally, such reduction in exposure to the environment can aid in preserving the self-adhesive nature of the flexible member 110.

Referring now to FIG. 3, there is shown a front perspective view of an embodiment of the pet treat pocket illustrating how the pet treat pocket can be folded to enrobe a pill. In the shown embodiment, a pill 140 can be inserted into a pocket 120 disposed on a top surface 115 of a flexible member 110. As the pocket 120 is dimensioned smaller than the flexible member 110, a lip 150 is formed and defined. The lip 150 can be physically manipulated to enrobe and surround the pocket 120 by folding the lip 150 up, over, and around the pocket 120, thereby encasing and securing the pill 140 within the pocket 120. In one embodiment, the lip 150 extends at least as wide as half the pocket width plus the pocket depth, such that when opposing sides of the lip 150 are folded over the pocket 120, the lip 150 fully encases the pocket 120. In some embodiments, the flexible member 110, and therefore the lip 150, is configured to adhere to itself. By nature of such self-adherence, once the pill 140 and pocket 120 are molded into a desired shape, such shape is held in place. In this manner, once the user lets go of the invention, for example by offering it to a pet, the flexible member 110 and lip 150 will not unravel and reveal the presence of the pill 140.

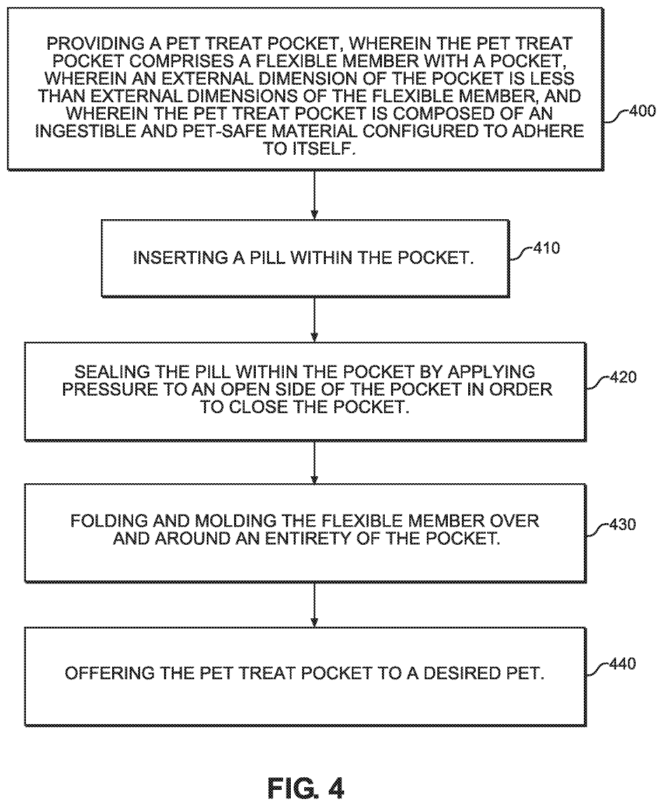

Referring now to FIG. 4, there is shown a chart of a method of use of an embodiment of the pet treat pocket. An object of the present invention is to provide a healthy treat for a pet, as well as to provide a mechanism through which a pill can be hidden and offered to the pet. As the invention can be a stand-alone treat, any mention of a pill and related steps, are optional in some embodiments, as the pet treat pocket can be offered without a pill. A method of use comprises the steps of first providing a pet treat pocket, wherein the pet treat pocket comprises a flexible member with a pocket, wherein an external dimension of the pocket is less than external dimensions of the flexible member, and wherein the pet treat pocket is composed of an ingestible and pet-safe material configured to adhere to itself 400. A pill is inserted into the pocket 410 and then the pill is sealed within the pocket by applying pressure to an open side of the pocket in order to close the pocket 420. The pressure applied causes the open side of the pocket to adhere to the flexible member by virtue of the self-adhesive properties of the ingestible and pet-safe material. Folding and molding the flexible member over and around an entirety of the pocket 430 ensures that the pill is contained therein, and any visual or other sensory cues as to the presence of the pill are masked, blocked, or similarly prevented. The final step is the offering of the pet treat pocket to a desired pet 440 for this ingestion and consumption.

It is therefore submitted that the instant invention has been shown and described in what is considered to be the most practical and preferred embodiments. It is recognized, however, that departures may be made within the scope of the invention and that obvious modifications will occur to a person skilled in the art. With respect to the above description then, it is to be realized that the optimum dimensional relationships for the parts of the invention, to include variations in size, materials, shape, form, function and manner of operation, assembly and use, are deemed readily apparent and obvious to one skilled in the art, and all equivalent relationships to those illustrated in the drawings and described in the specification are intended to be encompassed by the present invention.

Therefore, the foregoing is considered as illustrative only of the principles of the invention. Further, since numerous modifications and changes will readily occur to those skilled in the art, it is not desired to limit the invention to the exact construction and operation shown and described, and accordingly, all suitable modifications and equivalents may be resorted to, falling within the scope of the invention.

1) A pet treat pocket, comprising:
a flexible member including a pocket disposed on a top surface of the flexible member;
the pocket sized to encapsulate a pill within an interior volume of the pocket;
an exterior pocket length less than a member length, and an exterior pocket width less than a member width such that the flexible member extends beyond the exterior dimensions of the pocket thereby forming a lip;
the lip surrounding a circumference of the pocket;
the member and the pocket both composed of an ingestible and pet-safe material; and
the ingestible and pet-safe material configured to adhere to itself.

2) The pet treat pocket of claim 1, wherein the flexible member and the pocket is a single layer.

3) The pet treat pocket of claim 1, wherein the flexible member is a first layer and the pocket is a second layer disposed on a top surface of the flexible member.

4) The pet treat pocket of claim 1, wherein the lip is sized to completely enrobe an entirety of the pocket when the flexible member is molded over the pocket.

5) The pet treat pocket of claim 1, wherein the ingestible and pet-safe material is malleable by hand.

6) The pet treat pocket of claim 1, wherein the ingestible and pet-safe material is organic.

7) The pet treat pocket of claim 1, wherein the ingestible and pet-safe material is palatably flavored.

8) The pet treat pocket of claim 1, wherein the ingestible and pet-safe material is a thin gelatin-based material.

9) The pet treat pocket of claim 1, wherein the ingestible and pet-safe material comprises montmorillonite clay and a binding material.

10) The pet treat pocket of claim 1, wherein the ingestible and pet-safe material does not comprise calcium bentonite.

11) The pet treat pocket of claim 1, whereupon the pill being encapsulated by the pocket, the ingestible and pet-safe material blocks odors emanating from the pill from escaping the pocket.

12) The pet treat pocket of claim 1, whereupon the pill being encapsulated by the pocket, the ingestible and pet-safe material masks odors emanating from the pill from escaping the pocket.

13) The pet treat pocket of claim 1, wherein a plurality of pockets is disposed on the flexible member.

14) The pet treat pocket of claim 13, wherein at least one perforation separates at least one of the plurality of pockets from a neighboring pocket.

15) The pet treat pocket of claim 14, wherein seven pockets are disposed on the flexible member.

16) The pet treat pocket of claim 1, further comprising a backing material, the backing material removably secured to a rear surface of the flexible member.

17) A method of using a pet treat pocket to administer medication comprising the steps of:
providing a pet treat pocket, wherein the pet treat pocket comprises a flexible member with a pocket, wherein an external dimension of the pocket is less than dimensions of the flexible member, and wherein the pet treat pocket is composed of an ingestible and pet-safe material configured to adhere to itself;
inserting a pill within the pocket;
sealing the pill within the pocket by applying pressure to the open side of the pocket in order to close the pocket;
folding and molding the flexible member over and around an entirety of the pocket; and
offering the pet treat pocket to a desired pet.
